(19) United States
(12) Patent Application Publication
(10) Pub. No.: US 2026/0165660 A1
(43) Pub. Date: Jun. 18, 2026

(54) ARTIFICIAL INTELLIGENCE BASED NEURAL TEMPORAL FINGERPRINTING FOR PREDICTIVE DIAGNOSTICS

(71) Applicant: Adnexus Biotechnologies Inc.,, Dallas, TX (US)

(72) Inventors: SHUBHAM CHANDRA, Katy, TX (US); Gaurav Chandra, Dallas, TX (US); Kirsten Bischof, Cape Town (ZA); Kamlendra Singh, Columbia, MO (US)

(73) Assignee: Adnexus Biotechnologies Inc.,, Dallas, TX (US)

(21) Appl. No.: 18/979,873

(22) Filed: Dec. 13, 2024

(51) Int. Cl. A61B 5/00; A61B 5/0205; A61B 5/024; A61B 5/16; A61B 5/346; A61B 5/374; G16H 50/30

(52) U.S. Cl. CPC A61B 5/7267; A61B 5/0205; A61B 5/165; A61B 5/346; A61B 5/374; A61B 5/7203; A61B 5/725; G16H 50/30; A61B 5/02405

(57) ABSTRACT

The invention introduces a computerized method and system integrating Neural Temporal Fingerprinting with reward and penalty-based optimization for analyzing EEG and ECG data in predictive diagnostics. Utilizing advanced machine learning, including Convolutional and Recurrent Neural Networks, the system extracts temporal patterns and features from neural and cardiac signals. This enables accurate classification and prediction of conditions such as Alzheimer's, PTSD, epilepsy, arrhythmias, and cognitive impairments. Real-time or stored data is preprocessed to normalize signals, remove noise, and generate time-frequency representations like spectrograms. A hybrid deep learning model identifies spatial-temporal dependencies, while a reward-penalty mechanism prioritizes critical markers and reduces errors. Outputs include probability scores, actionable insights for clinicians, and personalized recommendations. Applications range from medical diagnostics and personalized healthcare to mental health monitoring and space research. The technology supports real-time monitoring via wearables, clinical systems, and telemedicine, offering scalable solutions to advance neurological and cardiovascular care.

### BACKGROUND TECHNICAL FIELD

The present disclosure relates to a method and computer-based apparatus for predicting outcomes based on EEG (Electroencephalography) and ECG (Electrocardiography) data. This method employs Neural Temporal Fingerprinting, a novel approach that utilizes advanced machine learning techniques to analyze and extract unique temporal patterns and features from EEG and ECG signals. The extracted fingerprints enable highly accurate predictions and assessments of physiological conditions, providing valuable insights into health, diagnostics, and usability over time. The method incorporates reward and penalty function-based optimization to refine predictive accuracy and enhance model performance. In lieu of this patent application and research, no federally/Government sponsored research or development-based funding was used.

### BACKGROUND OF THE INVENTION

EEG (Electroencephalography) and ECG (Electrocardiography) are essential tools for monitoring brain and heart activity. EEG captures neuronal electrical signals via scalp electrodes, aiding in diagnosing neurological disorders like epilepsy, Alzheimer's, and sleep issues, while also supporting neurofeedback, brain-computer interface research, and cognitive studies.

ECG records heart electrical activity through chest and limb electrodes, critical for diagnosing arrhythmias, heart attacks, and monitoring heart rate variability in chronic conditions. It is widely used in healthcare, wearable devices, and telemedicine for real-time cardiovascular insights.

Together, EEG and ECG provide complementary neural and cardiac data, enabling early diagnosis, personalized care, and improved health outcomes. Integration with AI enhances their capabilities, supporting predictive analytics, continuous monitoring, and precise diagnostics.

EEG plays a pivotal role in neurofeedback, brain-computer interface (BCI) research, and cognitive neuroscience. Neurofeedback trains individuals to regulate brain activity, aiding conditions like ADHD, PTSD, and anxiety. BCI applications use EEG to interpret brain signals, enabling communication and control for individuals with physical disabilities. EEG also studies brain function during tasks such as memory recall and decision-making. EEG and ECG are increasingly used to detect and monitor PTSD and neurological diseases. EEG identifies brainwave abnormalities, such as amygdala hyperactivity or prefrontal cortex underactivity, associated with PTSD. Targeted neurofeedback therapies use these insights to help patients regulate brain activity. ECG complements EEG by measuring heart rate variability (HRV), a marker of stress and emotional regulation. Together, they reveal critical brain-heart interactions, offering a holistic approach to diagnosis and monitoring. For other neurological diseases, EEG detects patterns like spike-and-wave discharges in epilepsy, disrupted theta rhythms in Alzheimer's, and hyperactive beta waves in anxiety disorders. ECG provides insights into cardiac irregularities linked to neurological conditions, enhancing diagnostics by correlating neural and cardiovascular responses. Therapeutically, EEG monitors treatment efficacy, while integration with ECG assesses impacts on both brain and heart health. Combined with AI, these modalities enable predictive modeling, real-time monitoring, and early detection of disease progression. Overall, EEG and ECG provide non-invasive, comprehensive insights into brain and heart activity, advancing neuroscience, improving diagnostics, and fostering innovative therapeutic strategies.

The 10-20 system is a standardized method for placing electrodes on the scalp during an EEG, ensuring consistency and reproducibility across different individuals and studies. Each electrode is named based on its location: frontal (F), central (C), parietal (P), occipital (O), temporal (T), and midline (Z for zero). Odd-numbered electrodes are positioned on the left hemisphere, while even-numbered ones are on the right. The electrodes are placed at 10% and 20% distances from specific anatomical landmarks on the head, such as the nasion (bridge of the nose) and inion (bony bump at the back of the head), to achieve uniform coverage of the scalp.

EEG channels are strategically positioned to correspond to specific brain regions, and their recorded activity can reveal insights into various neurological conditions. The frontal lobe, represented by channels such as F3, F4, F7, F8, AF3, and AF4, is commonly associated with conditions like epilepsy, ADHD, and depression. Frontal seizures are often detected here, while ADHD is linked to altered frontal activity patterns. Depression, on the other hand, may manifest as an asymmetry between the left and right frontal lobes.

The central region, captured by channels C3, C4, and Cz, is crucial for monitoring motor functions. It is often involved in disorders like Parkinson's disease and ALS, which affect motor cortex activity. The temporal lobes (T3, T4, T5, T6, F7, and F8) are integral for memory processing and are linked to temporal lobe epilepsy, Alzheimer's disease, and other memory disorders. Early-stage Alzheimer's often shows abnormal activity in these regions. The parietal lobe, with channels P3, P4, and Pz, is essential for cognitive functions and is often affected in Alzheimer's disease and dementia, where reduced parietal activity is a hallmark. The occipital lobe, monitored via O1 and O2, is associated with visual processing and conditions like visual epilepsies and migraine disorders, which involve altered occipital activity. Lastly, midline channels (Fz, Cz, Pz) often show generalized seizure activity and patterns associated with autism spectrum disorder (ASD).

TABLE 1
below summarizes the mapping of EEG channels to associated neurological disorders:

| Region | Channels | Associated Disorders |
|---|---|---|
| Frontal | F3, F4, F7, F8, AF3, AF4 | Epilepsy, ADHD, Depression, Anxiety |
| Central | C3, C4, Cz | Motor Disorders, ALS, Parkinson's |
| Temporal | T3, T4, T5, T6, F7, F8 | Temporal Epilepsy, Alzheimer's, Memory Disorders |
| Parietal | P3, P4, Pz | Alzheimer's, Dementia |
| Occipital | O1, O2 | Visual Epilepsy, Migraine Disorders |
| Midline | Fz, Cz, Pz | Generalized Seizures, Autism Spectrum Disorder |

While certain EEG channels are linked to specific disorders, accurate detection often requires advanced techniques such as spectrogram analysis, machine learning, and feature extraction. Modern EEG studies further enhance diagnostic capabilities by combining frequency analysis (e.g., alpha, beta, gamma waves) with spatial activity patterns to provide deeper insights into brain functions and neurological health.

EEG and ECG have profoundly impacted the fields of neuroscience and cardiology, revolutionizing how brain and heart functions are studied and understood. Their non-invasive capability to monitor real-time electrical activity has transformed diagnostics, enabling early detection and management of neurological and cardiac conditions. These technologies have also paved the way for advanced research, uncovering deeper insights into the complex workings of the brain and heart. Today, with the integration of AI, EEG and ECG have reached new heights, facilitating real-time analysis, predictive diagnostics, and personalized healthcare solutions. This progress builds on the pioneering efforts of early scientists, whose discoveries laid the foundation for these transformative tools. As fundamental instruments in modern medicine, EEG and ECG continue to inspire innovation in health technology, shaping the future of medical diagnostics and treatment. Modern techniques have significantly enhanced the precision of neurological disease detection using EEG, building upon the diagnostic clues provided by specific channels. Frequency analysis is a cornerstone of EEG interpretation, examining the brain's electrical activity across alpha, beta, delta, theta, and gamma frequency bands. Abnormal patterns, such as increased delta activity in dementia or reduced alpha waves in Alzheimer's, often indicate underlying neurological conditions. Machine learning and AI have revolutionized EEG analysis by processing complex patterns in multi-channel data to classify disorders with high accuracy. These technologies are particularly effective for the early diagnosis of conditions like Alzheimer's, epilepsy, and autism spectrum disorder (ASD). Another advanced method is spectrogram analysis, which visualizes the power of different frequencies over time, providing a detailed map of brain activity. This technique is invaluable for identifying focal seizures or detecting abnormal rhythms associated with various disorders. Finally, event-related potentials (ERPs) measure the brain's electrical responses to specific stimuli, offering insights into cognitive and sensory deficits. ERPs are particularly useful for assessing conditions that affect attention, perception, or memory, such as ADHD or traumatic brain injuries. Together, these advanced EEG techniques expand the capability to detect and understand a wide range of neurological diseases.

AI offers transformative potential for advancing EEG and ECG technologies by enabling accurate analysis, predictive modeling, and personalized healthcare applications. Various AI models are particularly suited for processing the time-series data generated by these technologies, uncovering complex patterns and insights. Convolutional Neural Networks (CNNs) excel at extracting spatial and temporal features from raw EEG and ECG signals, identifying abnormalities such as arrhythmias or seizures. Graph Neural Networks (GNNs), on the other hand, capture relationships and interactions between EEG channels or ECG leads, making them highly effective in modeling correlations and dependencies across different signal sources. The combination of CNNs and GNNs is especially powerful, as CNNs extract detailed local features from spectrograms or raw signals, while GNNs provide a broader context by modeling the spatial or relational structure of the data. This hybrid approach enables robust diagnostic tools capable of both feature extraction and contextual reasoning. Recurrent Neural Networks (RNNs) and Long Short-Term Memory (LSTM) networks are ideal for modeling sequential data, predicting trends like seizures or cardiac events. Emerging models such as Transformers analyze longer temporal dependencies with high accuracy, while Autoencoders help reconstruct signals, filter noise, and detect subtle anomalies. Traditional methods like Support Vector Machines (SVMs) remain effective for tasks like stress or sleep stage classification, particularly when working with smaller datasets, and Generative Adversarial Networks (GANs) can synthesize realistic EEG and ECG data to augment training sets and improve model robustness. Ensemble models, which combine multiple AI approaches such as CNNs, GNNs, and RNNs, and Bayesian Networks, which estimate uncertainties in predictions, further enhance the reliability and robustness of diagnostic tools in the field of EEG and ECG analysis.

These AI-driven advancements enable real-time monitoring of brain and heart activity, providing early warnings for conditions such as seizures or heart attacks. They also support personalized healthcare by tailoring insights and treatments to an individual's unique signal patterns. Predictive diagnostics powered by AI can detect neurological conditions like Alzheimer's or cardiac disorders before symptoms arise, while neurofeedback and biofeedback applications leverage EEG data for managing stress, anxiety, PTSD, and other conditions. Additionally, AI-powered wearable devices enable remote monitoring of heart and brain activity, advancing telemedicine and improving accessibility to care. By integrating AI models into EEG and ECG technologies, researchers and healthcare providers are unlocking new opportunities for diagnostics, monitoring, and treatment, paving the way for more efficient and personalized patient care.

Convolutional Neural Networks (CNNs) and Random Forests are two widely used AI models, each excelling in different domains of data analysis. CNNs are particularly effective for tasks involving spatial data, such as image and signal processing. They use convolutional layers to automatically extract features, such as edges, textures, or patterns, making them ideal for applications like medical imaging, object recognition, and time-series analysis. CNNs are highly scalable, capable of identifying complex patterns with minimal preprocessing, and are frequently used in advanced applications like detecting anomalies in EEG/ECG signals or classifying biomedical images. In contrast, Random Forests are ensemble learning models based on decision trees. They work by constructing multiple decision trees during training and outputting the majority vote or average prediction for classification and regression tasks. Random Forests are known for their robustness and interpretability, making them well-suited for tabular datasets and feature importance analysis. They perform exceptionally well in scenarios where data is less structured, such as predicting outcomes based on patient demographic data or identifying key predictors in large datasets. While CNNs shine in extracting hierarchical features from complex data, Random Forests are preferred for their simplicity and effectiveness in handling mixed-type variables and smaller datasets. Together, these models provide complementary capabilities for solving diverse AI-driven problems in healthcare, research, and beyond.

While neural networks are powerful for generating potential solutions, they do not always guarantee optimal or feasible results, particularly when complex physical or chemical constraints must be met. The reward and penalty function-based optimization method is a dynamic optimization methodology that refines machine learning models by systematically incorporating feedback into the training process. In this approach, the model is assigned rewards for accurate predictions or outputs that align with predefined target metrics, and penalties for errors, misclassifications, or deviations from expected results. This feedback-driven mechanism enables the model to iteratively adjust its parameters, such as weights and biases, optimizing its performance over successive training cycles. By emphasizing relevant patterns and reducing the impact of noise or irrelevant features, the model achieves greater precision and robustness.

This optimization methodology is particularly powerful when applied to complex datasets, such as time-series data in EEG and ECG analysis, where subtle and transient patterns are critical for accurate predictions. By integrating this reward-penalty system into the optimization process, the model not only enhances its ability to detect meaningful temporal features but also improves its adaptability to real-world variability and noise. As a result, this methodology serves as a foundation for building highly accurate, reliable, and scalable machine learning solutions across diverse applications.

The invention, which integrates Neural Temporal Fingerprinting (NTF) with a Reward and Penalty-Based Optimization framework, offers significant advancements over prior art patents that focus on the application of machine learning (ML) and artificial intelligence (AI) to analyze EEG and ECG data for neurological predictions. Traditional approaches often rely on static optimization techniques and predefined loss functions, limiting their adaptability and accuracy when applied to real-world datasets. In contrast, the invention incorporates a dynamic reward and penalty mechanism that adjusts model weights during training, enabling enhanced sensitivity and specificity, particularly for critical predictions such as seizures, Alzheimer's detection, and REM sleep analysis.

Prior art patents, such as those describing spatio-temporal pattern recognition or functional connectivity in EEG and ECG signals, typically use fixed architectures and static modeling approaches. For example, methods like Random Decision Forests or single-layer CNN architectures have been employed to predict neurological conditions. While effective within limited domains, these approaches often fail to generalize across diverse signal types or complex temporal dependencies inherent in EEG and ECG data. The invention addresses this gap by combining CNNs for spatial feature extraction with RNNs or LSTMs to capture intricate temporal relationships. This hybrid architecture outperforms the static models by enabling more nuanced pattern recognition across modalities.

Another key differentiation lies in the optimization strategy. Prior art solutions generally lack mechanisms to dynamically adapt to evolving patterns in new datasets or address imbalanced data distributions. The invention's reward and penalty framework overcomes these limitations by encouraging the model to prioritize critical classifications (e.g., seizure detection) while penalizing false positives or misclassifications. This dynamic scoring system not only enhances accuracy but also ensures robustness and reliability in high-stakes applications.

Furthermore, the invention extends its utility by integrating EEG and ECG data within a unified platform, allowing for cross-modality predictions. Prior patents have typically focused on either EEG or ECG analysis, with limited exploration of their combined diagnostic potential. The invention bridges this gap, enabling more comprehensive assessments of conditions influenced by both neural and cardiovascular factors, such as stress and sleep disorders.

In terms of applications, the invention expands its scope beyond the singular focus of many prior art patents, addressing neurological disorders, sleep cycle analysis, and neural conditions like anxiety and depression. This versatility makes it suitable for both clinical diagnostics and real-time monitoring in wearable devices. Unlike the static approaches in prior art, the invention's scalability and adaptability make it robust for diverse datasets and evolving patterns, setting a new benchmark in the field of ML/AI for bio-signal analysis.

The invention, which integrates Neural Temporal Fingerprinting (NTF) with a Reward and Penalty-Based Optimization framework, represents a significant advancement over prior art patents that utilize machine learning (ML) and artificial intelligence (AI) to analyze EEG (Electroencephalography) and ECG (Electrocardiogramata for neurological predictions. Unlike traditional approaches that rely on static optimization techniques and predefined loss functions, this invention incorporates a dynamic feedback mechanism that adjusts model weights during training, resulting in enhanced sensitivity and specificity for predictions related to seizures, Alzheimer's detection, and REM sleep analysis. This innovative framework not only addresses the limitations of prior methodologies but also establishes a robust foundation for extending its application to other neural diseases.

Building on this invention, we propose a hybrid methodology that expands its utility to Post-Traumatic Stress Disorder (PTSD) and other neural conditions. By integrating EEG and ECG data with heart rate variability (HRV) analysis, this hybrid approach provides a comprehensive understanding of brain-heart interactions. For PTSD, the methodology leverages precise temporal analysis of neuronal responses to predict brain reactions to trauma and identify specific triggers.

These insights are then integrated into neurofeedback therapies, empowering patients to modify their brain activity in real time and effectively reduce PTSD symptoms.

Beyond PTSD, the hybrid methodology demonstrates its versatility by addressing other neural diseases, such as anxiety disorders, epilepsy, and Alzheimer's. By combining neural biomarkers with cardiovascular data, it uncovers unique physiological patterns specific to each individual, enabling the development of personalized treatment plans. These plans offer targeted interventions while allowing for continuous monitoring of patient progress. Additionally, the methodology incorporates predictive modeling to anticipate individual responses to treatments, optimizing therapeutic strategies and outcomes.

This hybrid approach introduces key features that enhance its effectiveness across a range of applications. By combining EEG and ECG data, it captures the interplay between neural and cardiac activity, improving diagnostic accuracy for conditions influenced by stress, autonomic dysregulation, or cognitive decline. It develops predictive models that tailor treatments to each patient's unique physiological patterns, ensuring personalized and effective care. The integration of real-time neurofeedback with cardiac regulation empowers patients to manage their conditions proactively, fostering emotional resilience and symptom reduction. Furthermore, the hybrid methodology enables real-time monitoring through wearable devices and telemedicine platforms, providing clinicians with actionable data to evaluate treatment efficacy and adapt strategies dynamically.

By extending the capabilities of the core invention, this hybrid methodology offers a transformative solution for managing PTSD and a wide spectrum of other neural conditions. Its ability to combine Neural Temporal Fingerprinting with neurocardiac analysis bridges the gap between isolated neural or cardiac studies and holistic patient care. This innovation empowers clinicians with precise, data-driven insights and equips patients with tools to manage their conditions effectively, ultimately enhancing their quality of life and long-term outcomes.

### PRIOR ART

The field of neural and cardiovascular diagnostics has witnessed significant advancements, with various patents contributing novel methodologies for analyzing EEG (Electroencephalography) and ECG (Electrocardiogramata. These prior arts have introduced specialized frameworks, algorithms, and architectures to address specific challenges in detecting and predicting neurological and cardiac conditions. However, most approaches remain focused on single-modal data analysis, such as EEG for neural disorders or ECG for cardiac arrhythmias, limiting their scope in understanding complex brain-heart interactions. Additionally, traditional optimization techniques often lack the flexibility to handle dynamic dependencies in real-world data. This section compares the invention with notable prior arts, including U.S. Pat. No. 11,839,480B2, US20230190104A1, and US20210267474A1, highlighting their contributions and limitations relative to the invention's integrated and advanced multi-modal approach. The analysis aims to underscore how the invention addresses critical gaps in predictive diagnostics by combining EEG and ECG data, leveraging hybrid deep learning models, and incorporating a reward and penalty optimization framework for enhanced accuracy and adaptability.

Prior Art 1 (U.S. Pat. No. 11,839,480B2): This prior art focuses on neural signal processing using convolutional neural networks (CNNs) to detect abnormalities in EEG data for neurological conditions such as epilepsy and Alzheimer's disease. While effective for spatial feature extraction, it lacks the incorporation of additional physiological data like ECG signals. The optimization techniques in this prior art rely on static loss functions, limiting its adaptability and specificity in handling complex neural and cardiac interactions.

Prior Art 2 (US20230190104A1): This prior art emphasizes the processing of ECG data for detecting cardiac arrhythmias. It employs CNNs for feature extraction and focuses solely on cardiac conditions. However, it does not integrate neural data such as EEG, which is critical for conditions influenced by both brain and heart activity, such as PTSD. Furthermore, its machine learning framework is not designed to address dynamic dependencies or multi-modal integration.

Prior Art 3 (US20210267474A1): This prior art introduces a framework for neural fingerprinting, utilizing CNNs to analyze EEG data for neural disease prediction, including Alzheimer's and epilepsy. While it is adept at identifying neural biomarkers, it does not incorporate multi-modal data like ECG signals or leverage advanced optimization techniques. The architecture is focused on spatial analysis of EEG signals and lacks provisions for dynamic modeling of temporal dependencies.

The Invention: In contrast to the prior arts, the invention integrates both EEG and ECG data to predict neurological and cardiovascular conditions comprehensively. It utilizes a hybrid deep learning model that combines CNNs for spatial feature extraction, RNNs or LSTMs for capturing temporal dependencies, and Graph Neural Networks (GNNs) for modeling complex interdependencies between different data regions. The invention employs a reward and penalty optimization framework to refine its predictions, prioritizing critical features such as hyperactivity in specific brain regions, disrupted heart rate variability (HRV), and network-level interactions. It also supports real-time deployment in wearable devices and telemedicine platforms, making it adaptable for continuous health monitoring. By addressing the limitations of single-modal approaches in the prior arts, the invention provides a holistic and scalable diagnostic tool.

TABLE 2
Comparative Table

| | Prior Art 1 | | | |
|---|---|---|---|---|
| | (U.S. Pat. No. | Prior Art 2 | Prior Art 3 | |
| Aspect | 11,839,480B2) | (US20230190104A1) | (US20210267474A1) | The Invention |
| Signal | EEG and limited | ECG signals only | EEG signals only | EEG and ECG |
| Types | physiological | | | signals |
| | data | | | |
| Machine | CNNs for | CNNs for ECG | CNNs for EEG | CNNs for spatial |
| Learning | spatial patterns | feature extraction | analysis | patterns, |
| Architecture | | | | RNNs/LSTMs |
| | | | | for temporal |
| | | | | dependencies, |
| | | | | and GNNs for |
| | | | | interdependency |
| | | | | modeling |
| Optimization | Static loss | Static loss functions | Static loss functions | Reward and |
| Framework | functions | | | penalty |
| | | | | optimization |
| | | | | framework |
| Applications | Neurological | Cardiac arrhythmias | Neurological | Neurological |
| | disorders | | diseases | and |
| | | | | cardiovascular |
| | | | | conditions |
| Deployment | Clinical settings | Wearable and | Research-focused | Wearable |
| | | clinical devices | EEG analysis | devices, |
| | | | | telemedicine, |
| | | | | and continuous |
| | | | | health |
| | | | | monitoring |
| Multi- | Limited | None | None | Integrated EEG |
| modal | | | | and ECG |
| Integration | | | | analysis |
| Dynamic | Limited | No | No | Advanced |
| Modeling | | | | hybrid |
| | | | | modeling for |
| | | | | brain-heart |
| | | | | interactions, |
| | | | | including |
| | | | | GNNs |
| Predictive | Focus on neural | Focus on cardiac | Focus on predefined | Predicts brain- |
| Insights | biomarkers | biomarkers | neural biomarkers | heart |
| | | | | interactions, |
| | | | | hybrid |
| | | | | conditions, and |
| | | | | network-level |
| | | | | insights |

### SUMMARY OF THE INVENTION

The invention leverages Neural Temporal Fingerprinting combined with a Reward and Penalty-Based Optimization methodology to analyze EEG (Electroencephalography) and ECG (Electrocardiogramata for precise predictions related to neurological disorders, sleep cycles, and other neural conditions. Neural Temporal Fingerprinting identifies and extracts unique temporal patterns from EEG and ECG signals, such as brainwave activity and cardiac rhythms, enabling detailed assessments of physiological states. The integrated reward and penalty system dynamically refines the model's predictive capabilities by rewarding accurate detections of critical features, such as abnormal waveforms indicative of epilepsy or irregular heart rhythms associated with neurological conditions, while penalizing misclassifications or noise-induced errors. This approach ensures the model's adaptability and robustness, delivering highly accurate insights into conditions such as sleep disorders, stress, PTSD, and cognitive impairments. By combining advanced temporal analysis with a feedback-driven optimization process, the invention provides a powerful, non-invasive tool for advancing diagnostics, monitoring, and personalized treatment strategies in neuroscience and healthcare.

The prediction process begins with the collection of high-quality EEG and ECG data, ensuring minimal noise and artifacts during signal capture. The raw signals are preprocessed to remove unwanted noise, normalize the data for consistent scaling, and segment the signals into time windows suitable for temporal analysis. These prepared signals are then processed through a Convolutional Neural Network (CNN), which extracts hierarchical features, such as spikes in EEG signals or rhythm irregularities in ECG waveforms. CNN identifies localized patterns critical for detecting conditions like seizures or arrhythmias while using pooling layers to optimize computational efficiency by retaining essential information.

During the training phase, the model uses the Reward and Penalty-Based Optimization mechanism to iteratively refine its predictions. Accurate predictions are rewarded, reinforcing critical features, while errors or misclassifications are penalized, ensuring the model focuses on meaningful patterns. This feedback system dynamically adjusts the model's weights, improving accuracy and reducing the impact of noise. The iterative nature of this process enhances reliability and ensures the model converges efficiently. Once trained, the model is validated on unseen data to fine-tune its performance further.

In the prediction phase, the trained model processes new EEG and ECG signals, classifying them into predefined categories, such as normal, seizure, or arrhythmia, and generating probability scores for each condition. These predictions are contextualized with the patient's overall health and medical history, providing actionable insights for early diagnosis and personalized treatment plans. The system can also be deployed for real-time monitoring, integrated into wearable devices or telemedicine platforms, enabling continuous tracking of health parameters. By tailoring predictions to individual patient data, the system supports personalized care and improves therapeutic outcomes. Scalable and efficient, this methodology is well-suited for large datasets, diverse populations, and seamless integration into clinical workflows or consumer health applications, delivering accurate and actionable insights into neurological and cardiac health. As part of our invention, we are offering two approaches for the AI based Neural Temporal Fingerprinting for predictive diagnostics.

In Example 1, we demonstrate how seizure-related datasets can be processed and analyzed using machine learning techniques. This method combines data engineering (e.g., file processing and preprocessing) with advanced AI models such as Convolutional Neural Networks (CNNs). By extracting temporal and spatial features from EEG/ECG data, CNNs help create a robust predictive system tailored for healthcare applications, specifically for detecting seizures.

In Example 2, we extend the capabilities by adopting a hybrid approach that combines Convolutional Neural Networks (CNNs) and Graph Neural Networks (GNNs). This approach leverages the feature extraction power of CNNs alongside the relational modeling capabilities of GNNs, which analyze interactions between EEG/ECG channels as graph structures. By combining these strengths, the hybrid model achieves improved classification accuracy, offering a more comprehensive solution for analyzing EEG/ECG data and detecting other neurological diseases.

### DETAILED DESCRIPTION OF THE INVENTION

Neural Temporal Fingerprinting leverages advanced deep learning techniques to analyze EEG signals for diagnosing neurological conditions like epilepsy, Alzheimer's, and Parkinson's. This method involves segmenting EEG data into channels, applying temporal and spatial analyses, and using CNNs to detect and classify disease-specific neural patterns.

EEG signals, captured via the 10-20 electrode system, are segmented into channels (e.g., Fp1, Fp2, C3), representing activity in different brain regions. Temporal signals are converted into spectrograms for frequency-domain analysis, enabling dynamic neural activity visualization. Preprocessing filters noise, normalizes signals, and removes artifacts, ensuring clean data for feature extraction.

CNNs process spectrograms to extract hierarchical features, identifying disease markers such as rhythmic oscillations or transient spikes. Multi-channel configurations capture interdependencies across brain regions, while attention mechanisms highlight regions with abnormal activity. Features are fused for global analysis, and fully connected layers classify conditions like epileptic spikes or disrupted alpha rhythms in Alzheimer's.

A feedback-driven optimization framework assigns rewards for correct predictions and penalties for errors, refining the model iteratively. This ensures robust learning, efficiency, and adaptability to noisy datasets. Model performance is validated using metrics like accuracy and F1 score, while explainability tools like saliency maps enhance clinical interpretability.

Neural Temporal Fingerprinting combines temporal and spatial analysis with reward-penalty optimization, enabling early diagnosis, personalized treatment, and real-time monitoring of neurological diseases.

The reward function calculates how close the predicted value is to the center of the desired range for a property. The closer the prediction is to the center, the higher the reward (scaled between 0 and 100).

$$Reward ( x ) = max ( 0 , ( 1 - \frac{\vert x - center \vert}{range} ) ) \times 100$$

Where:
x is the predicted value

$$\begin{matrix}range = desired\_range [ 1 ] - desired\_range [ 0 ] , which is the width of the desired range \\ center = ( desired\_range [ 1 ] + desired\_range [ 0 ] ) / 2 , which is the midpoint of the desired ranged\end{matrix}$$

This equation rewards predictions that are closer to the center of the range, with a reward of 100 for predictions exactly at the center.

The penalty function is applied when the prediction falls outside the desired range. It imposes a quadratic penalty based on how far the prediction is from the acceptable range. The equation for the penalty function is:

$$Penalty ( x ) = \{ \begin{matrix}0 , & if desired\_range [ 0 ] \leq x \leq desired\_range [ 1 ] \\ {( desired\_range [ 0 ] - x )}^{2} , & if x < desired\_range [ 0 ] \\ ( x - {desired\_range [ 1 ]}^{2} , & if x > desired\_range\end{matrix}$$

### Where

x is the predicted value, desired_range [0] is the lower bound of the desired range, desired_range [1] is the upper bound of the desired range

The total score for the predictions is calculated as the balance between the weighted sum of rewards and the weighted sum of penalties for each property. This allows a balance between encouraging predictions close to the desired ranges (rewards) and penalizing large deviations (penalties).

The equation for the total score is:

$$Total Score = {\sum }_{properties} ( {w}_{prop} \times {Reward}_{{x}_{prop}} ) - {w}_{prop} \times Penalty ( {x}_{prop} ) )$$

Where: xₚᵣₒₚ is the predicted value for a property, wₚᵣₒₚ is the weight assigned to each property,
Reward(xₚᵣₒₚ) is the reward function applied to predicted value for that property prop.
Penalty (xₚᵣₒₚ) is the penalty function applied to predicted value for that property
If no weights are used, wₚᵣₒₚ is set to 1 for all properties

If weights are applied to prioritize certain properties over others, the weights are normalized so that the sum of all weights equals 1. The normalization equation is:

$${w}_{prop}^{′} = \frac{{w}_{prop}}{{\sum }_{properties} {w}_{prop}}$$

Where w′ₚᵣₒₚ is the normalized weight of that property
wₚᵣₒₚ is the original weight assigned to that property

$$The sum of all weights = 1.$$

These equations provide a framework for balancing rewards and penalties to optimize the predictions from the CNN model based on both desirable properties and real-world constraints.

This integrated framework offers numerous advantages, including increased diagnostic accuracy, reduced processing times, and enhanced adaptability. The combination of precise feature extraction by CNNs and feedback-driven optimization enables early detection of conditions like sleep apnea, arrhythmias, and cognitive disorders. Its scalability makes it suitable for deployment in wearable devices, telemedicine platforms, and clinical settings, while its personalization capabilities allow tailored predictions for individual patients, improving therapeutic outcomes. By uniting advanced neural network architectures with a dynamic optimization strategy, this methodology sets a new standard for the analysis of EEG and ECG data in neuroscience and cardiology.

Spectrogram analysis is a powerful technique for visualizing the frequency content of signals, such as EEG and ECG, over time. It represents a signal in a three-dimensional space: time, frequency, and amplitude (or power). This method is particularly effective for analyzing non-stationary signals, where frequency characteristics vary over time. It is ideal for applications such as neural disease diagnosis, sleep cycle analysis, and stress or PTSD monitoring, where understanding temporal and frequency changes in signals is crucial.

A spectrogram is generated by dividing a time-domain signal into overlapping segments, applying a window function to each segment, and computing the Fourier Transform for each windowed segment. This process results in a representation of how the signal's energy is distributed across different frequencies over time. Mathematically, the Short-Time Fourier Transform (STFT) is used to calculate the spectrogram. For a time-domain signal x(t), the STFT is defined as:

$$X ( t , f ) = {{\int }^{∞}}_{- ∞} x ( r ) w ( r - t ) {e}^{- j 2 π frdr}$$

Where X(t,f) is the complex-valued STFT x(r) is the time-domain signal w(r−t) is the window function centered at time t, and e^{-j2πfr} represents the Fourier Transform's complex exponential. The window function w(t), often as a Hamming, Hanning, or Gaussian window, minimizes spectral leakage by tapering the segment edges. The spectrogram, representing power spectral density, is then calculated as:

$$S ( t , f ) = {\vert X ( t , f ) \vert}^{2}$$

In practical applications, signals are discretized, and the spectrogram is computed as:

$$S [ n , k ] = {⌊ {\sum }_{m = 0}^{M - I} x [ m ] w [ m - n ] {e}^{- j 2 π km / M} ⌋}^{2}$$

Where S[n,k] is the spectrogram value at discrete time n and frequency k, M is the number of samples in the segment, and w[m−n] is the discrete window function.

Key components of spectrogram analysis include windowing, overlap, frequency bins, and color mapping. Window size M determines the tradeoff between time and frequency resolution. Short windows provide high time resolution but low frequency resolution, while longer windows offer the opposite. Overlapping adjacent segments (typically 50-75%) ensures continuity in the time-frequency representation. Frequency resolution is defined by the sampling rate f(s) and the segment size (M) as Δf=fₛ/M. The spectrogram's amplitude is often visualized using a color scale, where warmer colors represent higher power.

In EEG analysis, spectrograms are used to detect neural patterns, such as epileptiform discharges or abnormal slowing, and to capture rhythmic activities like alpha (8-12 Hz), beta (13-30 Hz), delta (0.5-4 Hz), and theta (4-8 Hz) waves. In ECG analysis, spectrograms help analyze cardiac rhythms, detect arrhythmic episodes, and reveal changes in heart rate variability associated with stress or PTSD. Spectrograms also enable dynamic monitoring of how neural or cardiac events evolve over time, making them invaluable for understanding transient phenomena.

Spectrogram analysis offers several advantages. It is particularly suited for non-stationary signals, providing an intuitive visualization of complex signal dynamics. The temporal and frequency features extracted from spectrograms can be fed into machine learning models, such as CNNs, for classification and prediction tasks. By combining spectrogram analysis with advanced machine learning techniques, these representations enable the detection of critical temporal and frequency features, delivering precise and actionable insights into neural and cardiac conditions.

### Advantages of the Invention

Neural Temporal Fingerprinting combined with Reward and Penalty-Based Optimization offers a groundbreaking method for analyzing EEG and ECG data, significantly enhancing the accuracy of predictions related to neurological disorders, sleep cycles, PTSD, and other neural and psychological conditions. Utilizing advanced machine learning to extract unique temporal patterns from EEG and ECG signals, this approach excels in detecting abnormalities linked to conditions like epilepsy, Alzheimer's, Parkinson's, cognitive impairments, and PTSD. The integrated reward and penalty system dynamically refines the model, emphasizing critical features indicative of these conditions while minimizing noise and misclassifications, ensuring robust performance across diverse patient populations and noisy datasets.

This method adapts to real-world variability, providing reliable insights across a broad spectrum of applications. For neurological disorders, it enables early detection of preictal states in epilepsy or early disruptions in alpha rhythms associated with Alzheimer's, facilitating timely interventions that can improve patient outcomes. In PTSD, it identifies stress-induced neural patterns and autonomic responses by analyzing EEG and ECG signals, offering actionable insights for managing stress and trauma-related episodes-making it invaluable for mental health professionals seeking objective biomarkers for diagnosis and monitoring. In sleep cycle analysis, it delivers detailed assessments by identifying changes in brainwave patterns and cardiac rhythms, offering valuable insights into conditions like insomnia, narcolepsy, and sleep apnea. These insights enhance understanding of sleep disruptions and their health implications. Its non-invasive nature makes it ideal for continuous and comfortable monitoring through wearable devices or telemedicine platforms, ensuring accessibility for long-term use in clinical and home settings.

Real-time processing capabilities further distinguish this methodology, enabling instant feedback on neural and cardiac activity. This feature is critical for real-time interventions in acute seizure episodes, cardiac irregularities, or PTSD-related autonomic spikes. Tailoring predictions to individual signal patterns enhances treatment personalization, optimizing interventions to align with each patient's unique physiological and psychological characteristics.

The methodology's versatility extends to stress monitoring, neurodegenerative disease progression, and cognitive load assessment. Its scalability allows deployment across clinical diagnostics, research, and consumer health technologies-supporting PTSD monitoring in veterans through wearable devices, stress detection in high-pressure environments, or neurodegenerative disease research.

By combining advanced feature extraction with feedback-driven optimization, Neural Temporal Fingerprinting provides a robust, efficient, and scalable solution for critical challenges in neuroscience, cardiology, and mental health. Its ability to deliver high accuracy, early detection, real-time insights, and personalized predictions positions it as a transformative tool for advancing diagnostic precision, improving treatment outcomes, and enhancing patient care across a wide range of applications.

### BRIEF DESCRIPTION OF THE DRAWINGS

Reference is made to the accompanying drawings in which an illustrative embodiment of the invention is shown, from which its novel features and advantages will be apparent.

FIG. 1 is a simplified workflow of the Neural Temporal Fingerprinting process. The Neural Temporal Fingerprinting method integrates seamlessly into the outlined workflow in the figure, providing a structured approach to learning temporal patterns in EEG or similar time-series data. The process begins with 1, Data Collection, where EEG signals from specific channels are recorded to capture intricate temporal features. This raw data is then subjected to 2, Preprocessing, involving noise filtering, normalization, and spectrogram generation to transform the signals into a format suitable for feature extraction.

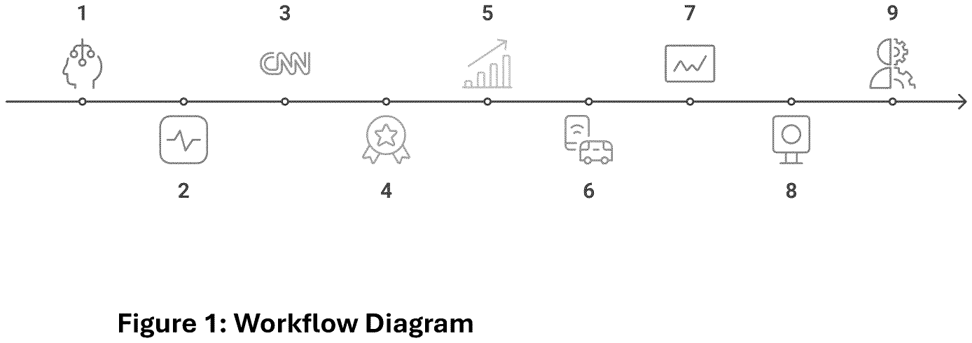

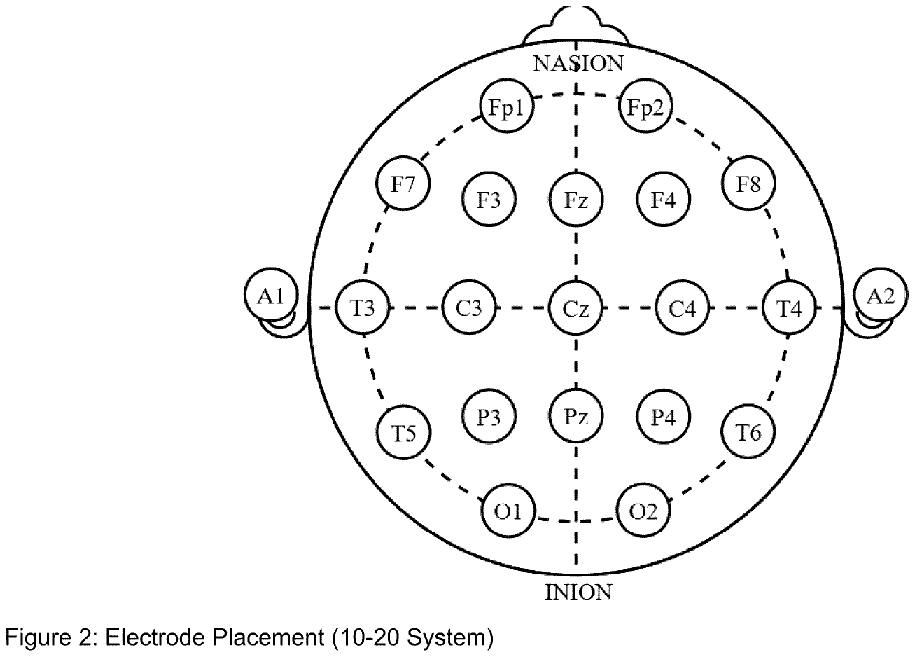

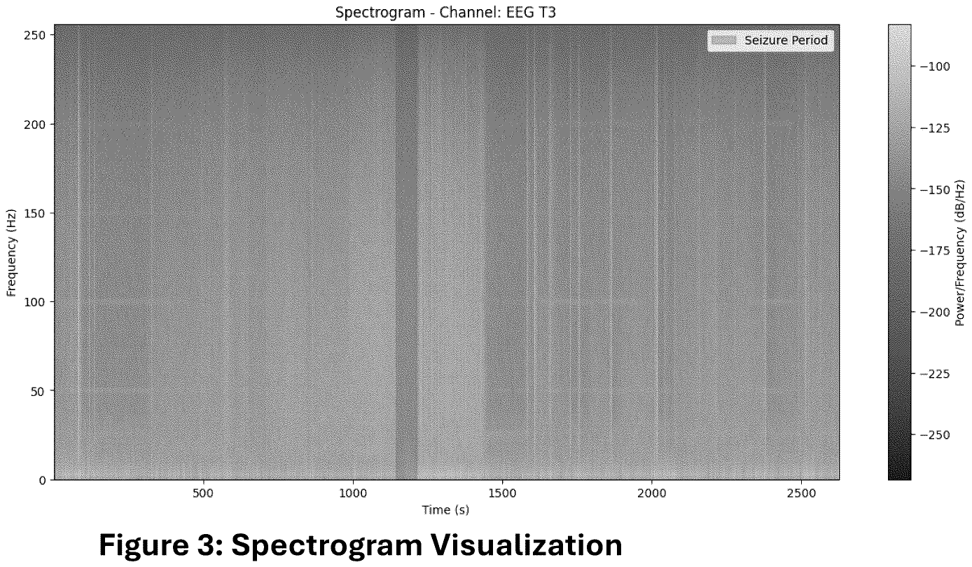

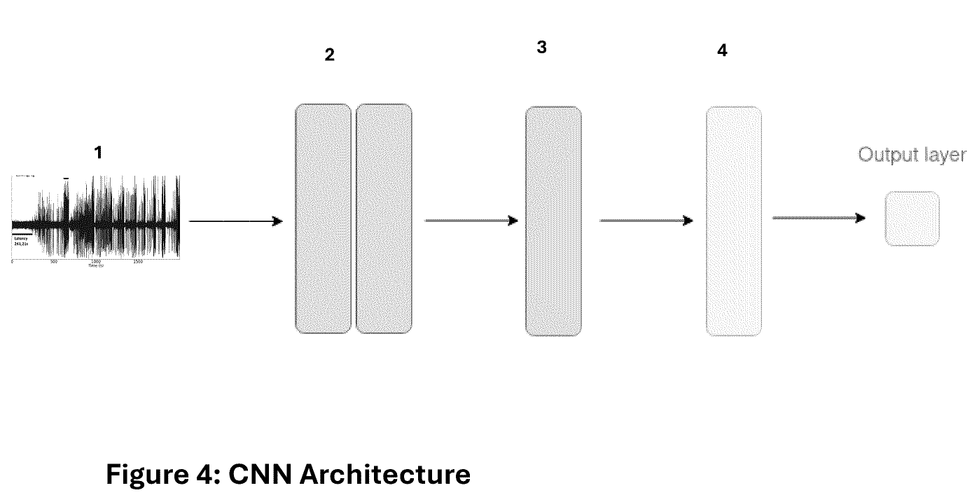

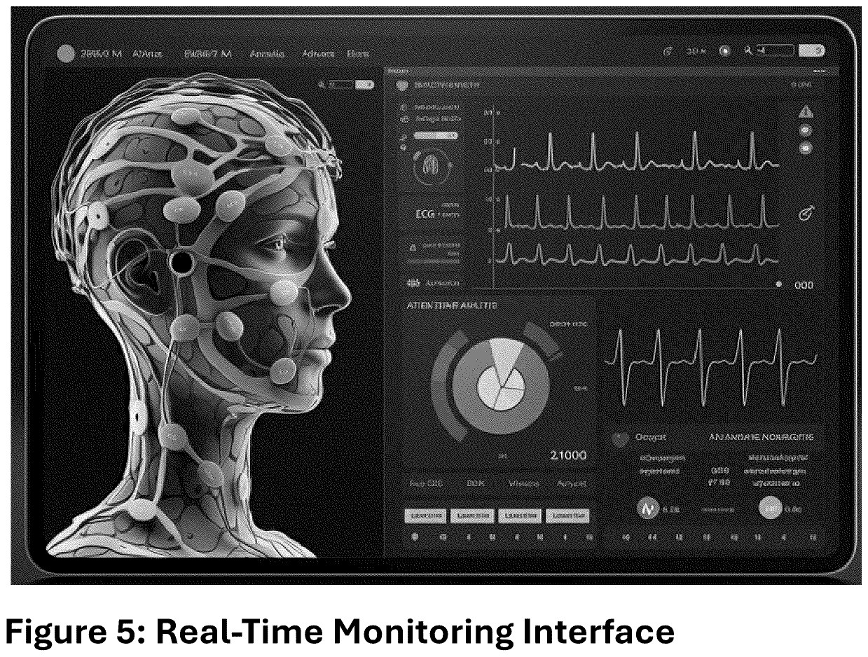

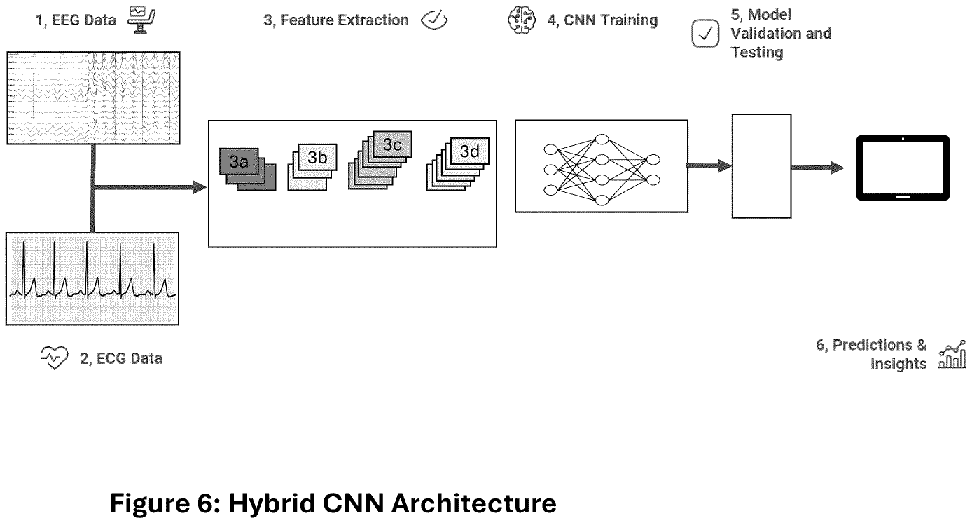

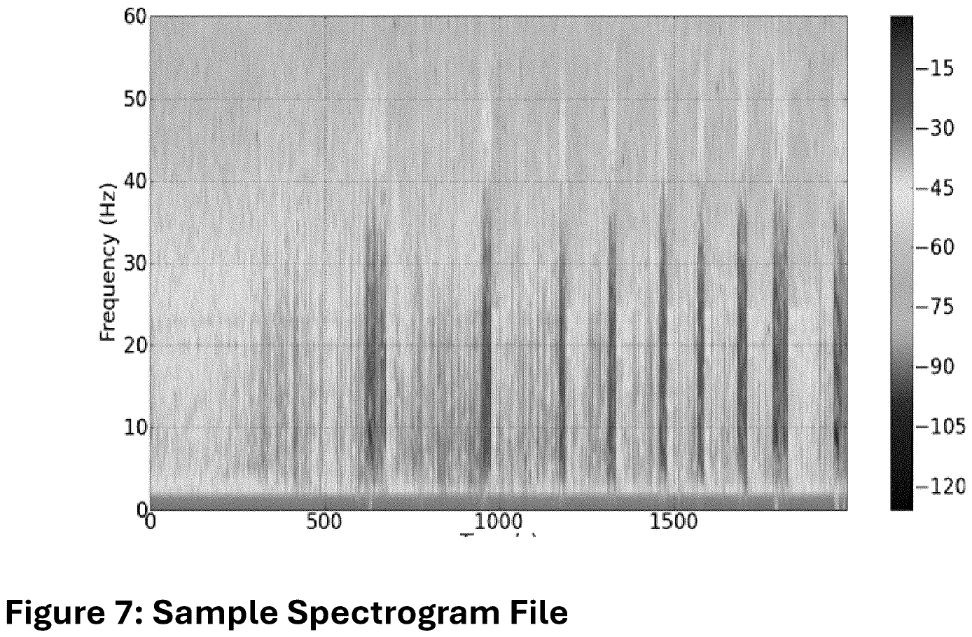

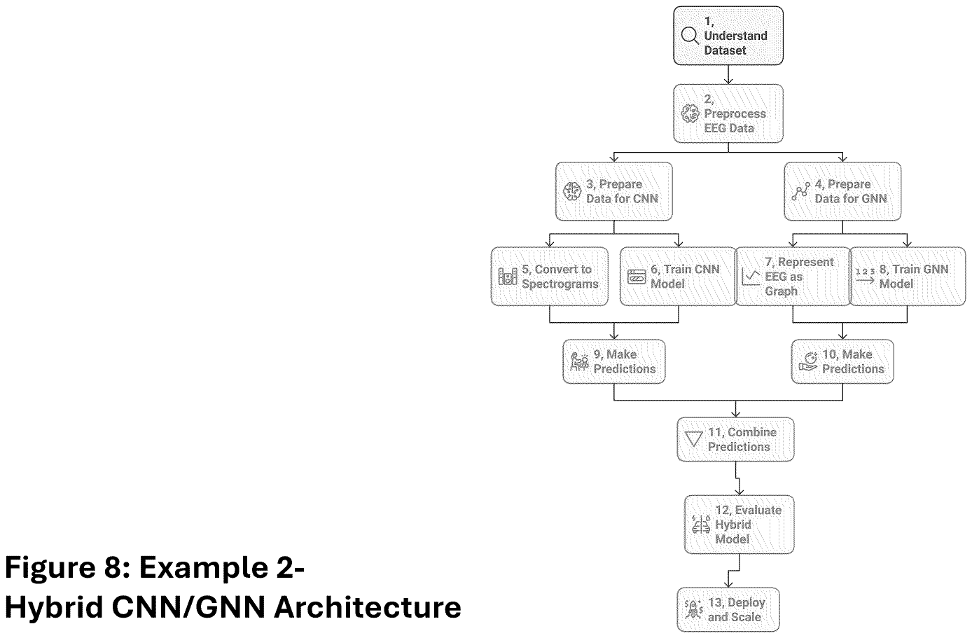

Using Feature Extraction via CNN, 3, the Neural Temporal Fingerprinting method leverages convolutional layers to detect and learn critical temporal and spatial patterns from the spectrograms. This automated feature extraction step is pivotal in identifying unique “fingerprints” that characterize specific conditions, such as Alzheimer's or epilepsy, by focusing on subtle but distinctive variations in the EEG signals.

In Model Training with Reward and Penalty-Based Optimization, 5, the Neural Temporal Fingerprinting framework employs a hybrid training strategy. A reward function incentivizes accurate predictions, while a penalty function discourages incorrect ones, dynamically guiding the model to higher accuracy and reliability. This iterative approach aligns the model with task-specific objectives, such as improved classification performance or sensitivity to early disease markers.

The trained model is then subjected to Validation and Iterative Improvement, 6, where its performance is evaluated on separate datasets, and refinements are made to enhance robustness. Upon achieving satisfactory results, the model is deployed for Prediction, 7, allowing it to analyze new EEG data and provide real-time insights into temporal patterns.

Post-deployment, Post-Prediction Analysis, 8, ensures the predictions are reliable and interpretable, helping identify areas for further refinement. For applications requiring dynamic adaptability, the method supports Real-Time Monitoring, enabling continuous analysis of EEG signals and adaptive learning in evolving environments. Finally, Personalization, 9, tailors the model outputs to specific user needs or clinical scenarios, ensuring the system provides actionable insights that align with individual requirements.

Neural Temporal Fingerprinting, as framed in this workflow, provides a comprehensive and iterative approach to developing robust EEG-based models. By combining feature extraction, hybrid optimization, and personalization, it effectively bridges the gap between raw temporal data and actionable insights.

FIG. 2 is a simplified illustration of 10-20 system. The 10-20 System is a standardized method used in electroencephalography (EEG) to place electrodes on the scalp for recording brain activity. This system ensures consistent electrode placement across different studies and clinical applications, enabling reproducibility and reliable comparisons. It is named after the proportional distances between electrodes, which are spaced at intervals of 10% or 20% of the total distance between anatomical landmarks on the scalp.

Key anatomical landmarks include:
Nasion: The depression at the bridge of the nose.
Inion: The bony prominence at the back of the skull.
Preauricular Points: Located just in front of the ears (A1 for the left ear, A2 for the right ear).

Electrodes are placed along specific lines connecting these landmarks, with positions identified by a combination of letters and numbers. The letters correspond to regions of the brain:
F: Frontal
T: Temporal
C: Central
P: Parietal
O: Occipital

The numbers differentiate the hemispheres:
Odd numbers (e.g., F3, T5) indicate the left hemisphere.
Even numbers (e.g., F4, T6) indicate the right hemisphere.
The letter “z” (e.g., Fz, Cz, Pz) denotes positions along the midline.

The figure illustrates a top-down view of the scalp, showing electrode positions like Fp1 and Fp2 (prefrontal), F7 and F8 (frontal), and O1 and O2 (occipital), among others. These positions correspond to underlying brain regions and are used to capture electrical activity related to various brain functions. The 10-20 system is widely used in clinical and research settings, including applications like diagnosing epilepsy, monitoring brain activity, and investigating cognitive processes. It provides a structured and universal framework for EEG measurements, enhancing the precision and reliability of brain signal recordings.

FIG. 3 illustrates a spectrogram of EEG data recorded from the T4 electrode, providing a visual representation of the signal's power distribution across time and frequency. The x-axis represents time in seconds, showing the temporal evolution of the EEG signal, while the y-axis denotes frequency in hertz, capturing the range of frequency components present in the signal. The color scale, measured in decibels (dB), encodes the power intensity at each time-frequency point, with yellow indicating regions of higher power and blue representing areas of lower power. This spectrogram is a valuable tool for analyzing temporal and spectral characteristics of EEG signals, enabling the identification of patterns or abnormalities that could be critical for tasks such as feature extraction and predictive modeling in neurological disease diagnosis.

FIG. 4 represents a basic architecture of a Convolutional Neural Network (CNN) for processing data, such as images. Here's a step-by-step explanation of the components shown:

### 1, Input Layer:

This is where the input data (e.g., an image) is fed into the network.
The input can be represented as a multi-dimensional array (e.g., height, width, and channels for an image).

### 2, Convolutional Layer:

This layer applies convolution operations to extract spatial features from the input data.
Filters or kernels slide across the input, detecting patterns such as edges, shapes, or textures.
Two stacked convolutional layers are shown here, indicating progressive feature extraction.

### 3, Max Pooling Layer:

This layer reduces the dimensionality of the feature maps from the convolutional layers.
It selects the maximum value from a defined window, retaining essential features while reducing computation.
The purpose is to downsample the data while preserving critical information.

### 4, Dense Layer:

Also known as a fully connected layer, it takes the flattened feature maps as input.
This layer integrates the features extracted by the convolutional and pooling layers to make a prediction or classification.

### 5, Output Layer:

This layer provides the final prediction or classification result.
For example, in an image classification task, the output could represent categories like “cat,” “dog,” or “bird.”
   The arrows indicate the flow of data through the layers, starting from the input image and progressing through feature extraction and classification steps to produce the output.

FIG. 5, represents a modern, interactive interface for monitoring and analyzing neural and cardiovascular activity using EEG (electroencephalography) and ECG (electrocardiogram of its components:

1. 3D Head Visualization with Electrodes:
The left side of the figure features a 3D anatomical model of a human head overlaid with electrode placements, likely adhering to the 10-20 system for EEG recording.
Electrodes are positioned on the scalp to capture electrical activity from specific brain regions, with labels denoting their placement.

### 2. Graphical Data Displays:

The right side of the interface displays multiple real-time signal graphs:
Top Graphs: EEG waveforms illustrating neural activity. Different colors are used to represent separate EEG channels or data streams.
Middle Graphs: ECG signals showing heart rhythms. Peaks and intervals are prominently visible, useful for identifying cardiac conditions like arrhythmias.
Additional smaller graphs or charts may show supplementary metrics or trends, such as attention spans or stress levels.

### 3. Circular Data Representation:

A circular chart in the bottom section visualizes specific metrics, possibly related to attention or cognitive state, in a percentage or comparative format.
It likely represents aggregated data for easy interpretation, such as focus levels or neural activity distribution.

### 4. Health Metrics and Analytics:

Textual and numerical indicators provide further insights, such as heart rate, neural biomarkers, or overall system status.
Alerts (indicated by symbols like exclamation marks) highlight anomalies or urgent conditions.

### 5. User-Friendly Interface:

The top menu offers navigation options, possibly for switching between different data modes (e.g., EEG, EMG, analytics).
Interactive tools like zoom, search, and filter icons ensure flexibility in data exploration and analysis.
   This figure effectively demonstrates how advanced systems can integrate real-time neural and cardiac data visualization, providing a comprehensive tool for diagnostic or research purposes. It showcases the potential for combining visual, numerical, and analytical insights in a user-friendly manner.

FIG. 6 illustrates the combined integration of EEG and ECG data feeds into a machine learning pipeline for feature extraction, CNN training, and predictions. The 1, EEG data feed represents brainwave activity captured through scalp electrodes, visualized as waveforms encompassing various frequencies like alpha, beta, and gamma waves. This data serves as the neural input to detect patterns associated with neurological conditions such as PTSD, stress, or epilepsy. Simultaneously, the 2, ECG data feed represents heart activity, particularly heart rate variability (HRV) and cardiac rhythms, shown as waveform peaks. ECG data provides critical cardiovascular insights, offering a comprehensive understanding of brain-heart interactions relevant to stress and anxiety.

The 3, feature extraction block processes and prepares both EEG and ECG data for CNN training. This involves several stages: 3a, preprocessing to remove noise and normalize the signals, 3b, converting EEG data into spectrograms and ECG data into HRV metrics, 3c, isolating patterns such as brainwave types (alpha, beta, gamma) and cardiac metrics (heart rate, HRV patterns), and 3d, aggregating these features into a unified dataset for training. The integration of EEG and ECG features ensures meaningful and holistic data inputs for the CNN.

The 4, CNN training block processes the combined EEG and ECG features to identify patterns, anomalies, or biomarkers associated with conditions like PTSD, anxiety, or cognitive impairments. This neural network develops a robust model capable of analyzing both data types with high accuracy. Following training, 5, the model validation and testing block evaluates the CNN model using unseen EEG and ECG datasets to ensure its ability to generalize and provide accurate predictions.

Finally, the 6, predictions and insights block generates actionable outputs by integrating EEG and ECG data analysis. This includes probability scores, classification results (e.g., PTSD markers), and recommendations tailored for clinicians or end-users. These insights provide a combined analysis of brain-heart dynamics for diagnosing or monitoring neurological and cardiovascular conditions. This figure underscores the synchronized role of EEG and ECG data in enhancing predictive model accuracy. By integrating these two data sources into a single machine learning pipeline, it demonstrates a holistic approach to healthcare applications, improving diagnostics, treatment, and overall patient outcomes.

FIG. 7 illustrates a spectrogram that visualizes the frequency content of EEG signals over time. This serves as an example to demonstrate the predictive capabilities of the algorithm trained on the generated training file. While a spectrogram is shown here, it is not strictly required for making predictions. When an EEG file is provided, the algorithm is designed to automatically process the data, convert it into a spectrogram if necessary, and generate predictions accordingly.

FIG. 8 represents a step-by-step workflow for implementing a hybrid CNN-GNN model to analyze EEG data and detect seizures. Here's an explanation of each step:

### 1. Understand Dataset

The process starts with understanding the dataset:
Input Format: EEG data stored in.edf files, containing multi-channel time-series signals.
Output Labels: Labels indicating whether a segment of EEG is a seizure or non-seizure.
Objective: To classify EEG segments accurately using a hybrid approach.

### 2. Preprocess EEG Data

This step involves preparing the raw EEG data for further processing:
Bandpass Filtering: Retain relevant frequency components (e.g., 0.5-50 Hz).
Segmentation: Divide the signals into smaller time windows (e.g., 256 samples).
Normalization: Standardize each segment to ensure consistent input ranges.

### 3. Prepare Data for CNN

Once the EEG data is preprocessed, it is prepared for the CNN pipeline:
Convert to Spectrograms (Step 5): Transform each EEG segment into a 2D spectrogram using Fourier Transform. These spectrograms are visual representations of the signal's frequency content over time.
Train CNN Model (Step 6): Train a CNN (e.g., EfficientNet) on the spectrograms to classify them as seizure or non-seizure.

### 4. Prepare Data for GNN

Simultaneously, the EEG data is prepared for the GNN pipeline:
Represent EEG as Graph (Step 7): Treat each EEG channel as a node in a graph and compute correlations between channels to form edges. Extract features for each node (e.g., mean amplitude).
Train GNN Model (Step 8): Train a GNN (e.g., Graph Convolutional Network) on this graph representation to classify the graph as seizure or non-seizure.

### 5-6: CNN Pipeline

Step 5: Convert each EEG segment into spectrograms, normalize, and resize them to match the CNN input shape (e.g., 224×224×3).
Step 6: Train the CNN model on the spectrograms using labeled data.

### 7-8: GNN Pipeline

Step 7: Represent the EEG data as a graph by:
   Treating channels as nodes.
   Computing an adjacency matrix based on correlations between channels.
   Assigning features to nodes (e.g., mean or variance of the signal).
Step 8: Train the GNN model on graph-structured data.

### 9-10: Make Predictions

Step 9 (CNN): Use the trained CNN model to predict seizure or non-seizure based on spectrograms.
Step 10 (GNN): Use the trained GNN model to predict seizure or non-seizure based on the graph representation of the EEG data.

### 11. Combine Predictions

The predictions from the CNN and GNN models are combined using an ensemble strategy:
Majority Voting: The final prediction is based on the class predicted by most models.
Weighted Averaging: Combine CNN and GNN probabilities to determine the final class.

### 12. Evaluate Hybrid Model

The hybrid model's performance is evaluated:
Metrics: Accuracy, precision, recall, and F1-score are used to assess the effectiveness of the hybrid approach.
Comparison: The hybrid model is compared to standalone CNN and GNN models to validate its advantages.

### 13. Deploy and Scale

Finally, the hybrid model is deployed and scaled for real-world applications:
Automation: Automate the pipeline to handle new EEG files seamlessly.
Scalability: Optimize the model for faster inference and deployment on cloud or edge devices.

### EXAMPLES

The invention will now be illustrated, but not limited, by reference to the specific embodiments described in the following examples.

### Example 1

In this example, the dataset used consists of EEG recordings collected from 14 patients at the Unit of Neurology and Neurophysiology of the University of Siena. The subjects included nine males, aged 25-71, and five females, aged 20-58. The patients were monitored using a Video-EEG system with a sampling rate of 512 Hz, following the international 10-20 electrode placement system. Additionally, most recordings included one or two EKG signals. The diagnosis of epilepsy and seizure classification were performed by an expert clinician based on the criteria established by the International League Against Epilepsy, ensuring accuracy through a comprehensive review of clinical and electrophysiological data. The recordings were obtained as part of a regional research project called PANACEE, which aimed to develop low-cost, non-invasive, patient-specific devices for predicting epileptic seizures. Ethical approval for the study was granted by the University of Siena's Ethical Committee in accordance with the Declaration of Helsinki. All patients provided informed written consent, permitting video recording and the use of their data for scientific research purposes. The dataset is organized into 14 folders, each corresponding to a specific patient. The data is stored in EDF (European Data Format) files, with each folder containing between one and five EDF files (maximum size of 2.11 GB each) and a text file with metadata about the recordings and seizures. A supplementary CSV file, subject_info.csv, provides details such as patient demographics, seizure classifications, EEG channel count, seizure count, and total recording time in minutes. The database contains a total of 47 seizures over approximately 128 hours of recordings. The metadata also includes detailed information about seizure events, including their start and end times (formatted as hours.minutes.seconds), sampling rate, and the channels used for EEG and EKG signal extraction. This structured dataset provides a valuable resource for developing and testing algorithms for the prediction and detection of epileptic seizures.

### Step-by-Step Workflow for Seizure Data Processing and Analysis

### 1. Mounting Google Drive:

The workflow starts by mounting Google Drive to access stored files and datasets. This is essential to read input files, save processed data, and use pre-trained models efficiently.

### 2. Defining a Function to Process Seizure Data:

A custom Python function is created to process seizure-related text files. This function reads all .txt files in a specified folder and its subfolders.
It extracts important details such as:
File name, Registration start time, Seizure start time, Seizure end time
The function organizes this data and appends it to a master list.

### 3. Parsing Files for Seizure Information:

The function iterates through each line in the text files, looking for keywords like File name:, Registration Start Time:, Seizure Start Time:, and Seizure End Time:.
Extracted data is stored in variables and added to the output dataset.

### 4. Combining and Saving Data:

All extracted information from individual files is compiled into a single output file, which serves as the processed dataset for the next stages.
Example Code Snippet:

### 5. Preprocessing for Machine Learning:

After data extraction, preprocessing steps are applied to format the dataset for training. This may include:
Cleaning data, Normalizing time values, Encoding labels
The dataset is then split into training and testing sets.
   6. Feature Extraction from EEG Data:
If the workflow involves EEG data, spectrograms or time-series features may be extracted to represent seizure patterns effectively.

### 7. CNN Model Training:

A convolutional neural network (CNN) is used to analyze the processed data. The model is trained to classify patterns associated with seizures, enabling it to predict seizures from new data.
The model includes the following techniques:
   Data augmentation, Reward and penalty functions to guide training toward higher accuracy.

### 8. Evaluation and Prediction:

The trained model is evaluated using testing data to assess its accuracy, precision, and recall. Predictions are generated for new or unseen data to determine its effectiveness.

### 9. Saving the Model and Outputs:

The final trained model is saved as a file (e.g., .keras) for future use. Processed outputs are stored for reporting and further analysis.

### Example Explanation

This workflow is a practical example of how seizure-related datasets can be processed and analyzed using machine learning. The method demonstrates combining data engineering (file processing) with advanced AI techniques (CNNs) to create a robust predictive system for healthcare applications. This information is being moved to the Appendix for the Specifications.

In Table 3, the results from evaluation of FIG. 7
(sample) using the EPILEPSY_V2.keras are presented.

| Label | Probability (%) | Prediction |
|---|---|---|
| Seizure | 95.0383606 | Yes |
| No-seizure | 4.961639404 | No |

### Example 2

This example demonstrates a hybrid approach that combines Convolutional Neural Networks (CNNs) and Graph Neural Networks (GNNs) for analyzing EEG data to improve seizure detection accuracy. The methodology processes EEG data stored in .edf files to classify segments as seizure or non-seizure. CNNs are utilized to extract spatial and temporal features from spectrograms, effectively handling 2D visual representations derived from EEG signals. GNNs complement this by modeling relationships between EEG channels, capturing electrode interactions as graph structures to enhance classification accuracy. The process begins with data preprocessing, where EEG signals are loaded, bandpass-filtered (0.5-50 Hz), segmented into windows (e.g., 256 samples), and normalized for consistency. In the CNN pipeline, EEG segments are converted into spectrograms using Fourier Transform, resized, and classified using a pre-trained CNN model like EfficientNet. Meanwhile, the GNN pipeline represents EEG channels as nodes and their correlations as edges, creating a graph structure. Features such as mean amplitude are extracted for each channel, and a GNN model is applied to classify the graph as seizure or non-seizure. During prediction, the CNN and GNN models independently classify the EEG data. Their outputs are combined using methods like majority voting or weighted averaging to produce the final classification. This hybrid approach leverages the strengths of CNNs in spatial feature extraction and GNNs in modeling inter-channel relationships, resulting in more robust predictions. The hybrid model demonstrates consistent improvement in accuracy during training, achieving a peak test accuracy of 96.66% across several epochs and stabilizing above 94% by epoch 30. This performance surpasses standalone CNN or GNN models, highlighting the benefits of combining spatial and relational data analysis. Key metrics, including accuracy, precision, recall, and F1-score, validate the model's effectiveness. In addition to its high accuracy, the hybrid approach offers flexibility through its modular design, allowing independent tuning of the CNN and GNN components. GNN also enhances interpretability by providing insights into channel relationships, complementing the CNN's feature extraction. Implementation involves using tools like pyedflib for .edf file handling, scipy for signal processing, torch_geometric for GNNs, and tensorflow.keras for CNNs. Predictions and results are saved in an Excel file for further analysis. This hybrid CNN-GNN approach not only excels in seizure detection but is also adaptable to other biomedical signal processing tasks that require the analysis of both spatial and relational data. Its combination of accuracy, flexibility, and interpretability makes it a valuable tool for advancing biomedical diagnostics. Table 4 provides the test accuracy of this model during the different epochs.

TABLE 4
TEST ACCURACY OF CNN/GNN HYBRID MODEL

| | Test Accuracy |
|---|---|
| Epoch | (%) |
| 1 | 87.54 |
| 2 | 85.41 |
| 3 | 89.97 |
| 4 | 91.49 |
| 5 | 91.49 |
| 6 | 89.97 |
| 7 | 90.27 |
| 8 | 91.49 |
| 9 | 91.79 |
| 10 | 92.10 |
| 11 | 91.49 |
| 12 | 92.40 |
| 13 | 93.31 |
| 14 | 91.79 |
| 15 | 93.92 |
| 16 | 90.88 |
| 17 | 93.31 |
| 18 | 92.40 |
| 19 | 91.79 |
| 20 | 91.49 |
| 21 | 93.31 |
| 22 | 92.40 |
| 23 | 91.79 |
| 24 | 93.92 |
| 25 | 93.31 |
| 26 | 95.14 |
| 27 | 94.83 |
| 28 | 91.49 |
| 29 | 94.22 |
| 30 | 95.44 |
| 31 | 95.74 |
| 32 | 95.74 |
| 33 | 94.83 |
| 34 | 96.66 |
| 35 | 96.66 |
| 36 | 96.05 |
| 37 | 94.22 |
| 38 | 96.05 |
| 39 | 96.05 |
| 40 | 96.05 |
| 41 | 93.92 |
| 42 | 96.66 |
| 43 | 93.92 |
| 44 | 95.44 |
| 45 | 93.92 |
| 46 | 95.44 |
| 47 | 96.05 |
| 48 | 95.74 |
| 49 | 95.44 |
| 50 | 94.53 |

TABLE 5
PREDICTIONS OF CNN/GNN HYBRID MODEL VS CNN MODEL
Two EEGs (in EDF format) were uploaded, and the
GNN/CNN and CNN model-based training files were
used to predict if the EEGs had seizures (1—True,
0—False). Both training models accurately predicted
the EEGs had signs of seizure activity.

| File | GNN/CNN_Prediction | CNN_Prediction |
|---|---|---|
| PN13-2.edf | 1 | 1 |
| PN13-3.edf | 1 | 1 |

### Commercial Applications

Neural Fingerprinting of EEGs and ECGs holds immense potential across a diverse range of applications, including medical diagnostics, preventive healthcare, biofeedback systems, therapy monitoring, and space research. By offering real-time, personalized, and actionable insights, this technology is driving innovation and revolutionizing multiple industries. Below are the key commercial applications:

### 1. Medical Diagnostics

Neurological Disorders:
   Enables early detection of conditions like epilepsy, Alzheimer's disease, and sleep disorders.
   Facilitates diagnosis of neurodegenerative diseases such as Creutzfeldt-Jakob Disease (CJD) and Parkinson's disease.
Mental Health and Cognitive Assessment:
   Evaluates brain activity to monitor cognitive load, stress levels, and mental health conditions such as anxiety and depression.
   Detects biomarkers for disorders like ADHD and PTSD.
Cardiovascular Health:
   Supports prediction and early diagnosis of heart conditions, including arrhythmias, myocardial infarctions, and heart failure.
   Allows continuous monitoring of ECG patterns for patients with chronic cardiac conditions.

### 2. Preventive Healthcare

Personalized Monitoring:
   Tracks EEG and ECG signals to identify at-risk individuals and enable early intervention.
   Creates unique neural and cardiac fingerprints for personalized health insights.
Lifestyle Integration:
   Embeds technology into wearable health devices for real-time assessments.
   Adapts health recommendations to individual physiological patterns, promoting tailored wellness strategies.

### 3. Biofeedback and Usability Applications

Wearable Technology Optimization:
   Enhances user experiences in devices like sleep trackers, fitness monitors, and stress management tools.
   Integrates real-time EEG and ECG analysis for adaptive feedback systems, improving usability.
Therapeutic Applications:
   Supports biofeedback-based therapies for conditions like PTSD, anxiety disorders, and chronic stress.
   Facilitates cognitive and physical rehabilitation through real-time physiological monitoring.

### 4. Therapy Monitoring

Medication Effectiveness:
   Analyzes EEG and ECG patterns to evaluate the impact of medications on neural and cardiac activity.
   Identifies neural markers to assess drug efficacy in neurological and cardiac treatments.
Rehabilitation and Recovery Tracking:
   Monitors recovery progress in post-surgical care and rehabilitative therapy.
   Tracks changes in physiological patterns to measure condition progression or remission over time.

### 5. Space Research

Stress and Adaptation in Microgravity:
   Monitors physiological impacts of stress and anxiety in microgravity environments, including changes in heart rate variability (HRV) and brainwave activity.
   Provides real-time insights into adaptation challenges, such as vestibular disturbances and mental fatigue.
Astronaut Health Monitoring:
   Tracks stress levels, sleep quality, and cognitive performance during space missions using EEG and ECG data.
   Enables early intervention through personalized biofeedback systems to maintain mental and physical well-being.
Predictive Modeling:
   Develops adaptive AI models to predict long-term physiological changes during extended space travel.
   Supports mission planning by identifying potential stressors and their mitigation strategies, ensuring optimal performance in extreme environments.

### 6. Research and Development

Clinical Research:
   Facilitates longitudinal studies to uncover trends in neural and cardiac health over time.
   Aids in developing predictive models for disease progression using neural fingerprints.
Healthcare Innovations:
   Drives the creation of advanced algorithms for real-time physiological signal analysis.
   Supports hybrid AI-driven solutions for adaptive and predictive healthcare systems, blending neural fingerprinting with other medical technologies.
      By integrating neural and cardiac insights into healthcare, wearable devices, and space exploration, Neural Fingerprinting of EEGs and ECGs is paving the way for a more personalized and predictive approach to human health. Its applications extend beyond diagnostics and therapy to include wellness, preventive care, and space research, making it a cornerstone technology for the future of health and innovation.

### Clinical Applications

Neuronal Temporal Fingerprinting (NTF) Applications in Neurodegenerative Diseases Neurodegenerative Diseases:

Alzheimer's Disease (AD) and Mild Cognitive Impairment (MCI):

Diagnosis: NTF can detect subtle changes in brain electrophysiology before structural changes appear on imaging. This early detection can differentiate between AD, MCI, and normal aging, facilitating earlier intervention.

Monitoring: By tracking changes in EEG patterns over time, NTF can monitor disease progression, cognitive decline, and response to treatments like new medications or cognitive therapies.

Prognostication: The patterns could predict disease severity or the likelihood of progression from MCI to AD, aiding in personalized treatment plans.

Parkinson's Disease (PD):

Cognitive Assessment: NTF could provide real-time, continuous monitoring of mental functions, capturing fluctuations not easily detected by conventional neuropsychological tests.

Therapeutic Feedback: It might help to tune closed-loop neurostimulation devices by providing immediate feedback on cognitive states, potentially enhancing the effectiveness of treatments like deep brain stimulation.

Huntington's Disease (HD):

Early Detection: NTF can be particularly useful in environments with limited resources for costly diagnostics. It provides a cost-effective biomarker through EEG analysis complemented by ECG for a broader physiological context.

Disease Monitoring: It could track the progression of motor, cognitive, and behavioral symptoms, aiding in adjusting therapeutic approaches.

Seizure Prediction and Detection: NTF can predict seizure onset by analyzing temporal patterns, allowing for preventive measures or timely emergency responses.

Interictal Period Analysis: It can identify neurocardiac dysfunctions during periods without overt seizures but with underlying electrical abnormalities, potentially preventing sudden cardiac events.

Application of Concurrent EEG and ECG monitoring in Migraines:

Biomarker Identification: NTF could help identify unique EEG signatures during both ictal and interictal phases, differentiating migraine from other headache disorders.

Personalized Therapy: By understanding the neurophysiological profile of a migraine attack, treatments could be tailored more effectively to individual EEG patterns.

### Neuropsychiatric Applications:

Application of Concurrent EEG and ECG monitoring in PTSD and ASD:

Biomarker Discovery: NTF could pinpoint specific EEG patterns associated with PTSD or ASD, facilitating diagnosis and the development of neurofeedback treatments.

Therapeutic Monitoring: It can assess the effectiveness of therapeutic interventions by observing changes in brain wave patterns.

### ADHD:

Risk Stratification: With NTF, one could identify children at higher risk for epilepsy due to EEG abnormalities, allowing for better management and treatment planning.

Application of Concurrent EEG and ECG monitoring in Traumatic Brain Injury (TBI):

Immediate Diagnosis: NTF can detect brain injury more sensitively than clinical examination alone, crucial for timely intervention in emergency settings.

Monitoring Recovery: It can track brain recovery or deterioration, aiding in decisions regarding weaning from coma or other intensive treatments.

1. A computerized hybrid method for predicting neurological and cardiovascular conditions, comprising acquiring real-time EEG signals via scalp electrodes or processing pre-recorded EEG data to monitor brain activity; acquiring real-time ECG signals or processing pre-recorded ECG data to monitor cardiac rhythms and heart rate variability (HRV); preprocessing EEG and ECG data by filtering artifacts, normalizing amplitudes, and segmenting signals into relevant time windows; transforming EEG data into spectrograms and extracting HRV metrics from ECG data; applying a hybrid deep learning model combining CNNs for spatial feature extraction, RNNs or LSTM networks for temporal dependencies, and GNNs for modeling inter-channel correlations in EEG and ECG data; employing a reward and penalty optimization mechanism to refine predictive accuracy; and generating diagnostic outputs including probability scores for neurological and cardiovascular conditions and actionable insights for clinicians.

2. The computerized hybrid method of claim 1, wherein the hybrid model correlates EEG alpha and beta wave activity with ECG HRV metrics to predict conditions such as stress, anxiety, and PTSD.

3. The computerized hybrid method of claim 1, wherein the system identifies brain-heart synchronization abnormalities linked to sleep disorders, PTSD, or cognitive decline.

4. The computerized hybrid method of claim 1, further comprising deployment in wearable devices for real-time health monitoring.

5. The computerized hybrid method of claim 1, wherein preprocessing includes bandpass filtering and segmentation of EEG and ECG signals to retain relevant frequency components and remove noise.

6. A computerized hybrid method for detecting neurological conditions, comprising acquiring EEG signals and preprocessing the data by filtering artifacts, normalizing amplitudes, and transforming the signals into spectrograms; applying CNNs to extract spatial and temporal features indicative of neurological abnormalities; utilizing GNNs to analyze spatial relationships and inter-channel dependencies in EEG data; employing a reward and penalty optimization mechanism to enhance predictive accuracy; and generating diagnostic outputs including probability scores for conditions such as epilepsy, Alzheimer's disease, and Parkinson's disease.

7. The computerized hybrid method of claim 6, wherein EEG spectrograms capture alpha, beta, gamma, theta, and delta wave activity for identifying specific neurological biomarkers.

8. The computerized hybrid method of claim 6, wherein CNNs incorporate attention mechanisms to prioritize regions exhibiting pathological activity such as epileptiform discharges.

9. A computerized hybrid method for detecting cardiovascular conditions, comprising acquiring ECG signals and preprocessing the data by filtering artifacts and extracting HRV metrics; applying CNNs to detect spatial and temporal patterns indicative of cardiac abnormalities; utilizing GNNs to model inter-lead correlations and enhance feature extraction; employing a reward and penalty optimization mechanism to refine predictive accuracy; and generating diagnostic outputs including risk assessments for arrhythmias, myocardial infarction, and heart failure.

10. The computerized hybrid method of claim 9, wherein preprocessing includes calculating HRV ratios to analyze stress and autonomic dysfunction.

11. The computerized hybrid method of claim 9, wherein CNNs identify irregular QRS complexes and T-wave abnormalities.

12. A computerized hybrid method for detecting PTSD, comprising acquiring EEG and ECG signals, preprocessing them to isolate neural and cardiac markers, and transforming EEG signals into spectrograms; applying a hybrid deep learning model combining CNNs, RNNs, and GNNs to analyze spatial and temporal dependencies in EEG and ECG data; employing a reward and penalty optimization mechanism to prioritize detection of PTSD-specific markers, including hyperactivity in the amygdala, disrupted HRV patterns, and emotional dysregulation; and generating diagnostic outputs and actionable recommendations for clinicians or therapists.

13. The computerized hybrid method of claim 12, further comprising neurofeedback integration to assist patients in regulating brain activity associated with PTSD symptoms.

14. The computerized hybrid method of claim 12, wherein the system is deployed in wearable devices for continuous PTSD monitoring in high-stress environments.

15. A computerized system for monitoring neural and cardiac activity, comprising sensors for acquiring real-time EEG and ECG signals; a preprocessing unit to filter noise, normalize signals, and segment data; a hybrid machine learning model combining CNNs, RNNs, and GNNs for analyzing neural and cardiac signals; a visualization interface for real-time and retrospective analysis; deployment options for integration with wearable devices, clinical systems, or telemedicine platforms; and a reward and penalty optimization mechanism to enhance model accuracy.

16. The computerized system of claim 15, wherein the hybrid model detects neurological and cardiovascular conditions, including Alzheimer's disease, epilepsy, PTSD, and arrhythmias.

17. The computerized system of claim 15, wherein GNNs model inter-channel correlations in EEG data and inter-lead relationships in ECG data for improved classification accuracy.

18. The computerized system of claim 15, further comprising real-time feedback to assist in managing acute episodes such as seizures or cardiac irregularities.

19. A computerized hybrid method for modeling EEG and ECG data, comprising representing EEG data as spectrograms and EEG channels as graph nodes for spatial and temporal analysis; representing ECG signals as graphs with leads as nodes to capture inter-lead correlations; combining CNN features and GNN embeddings in a unified classification framework; and employing attention mechanisms within the hybrid model to enhance predictions by focusing on regions or leads exhibiting significant abnormalities.

20. The computerized hybrid method of claim 19, wherein the hybrid model integrates attention mechanisms to enhance predictions by focusing on regions or leads exhibiting significant abnormalities.
